(19) United States
(12) Patent Application Publication
(10) Pub. No.: US 2014/0362199 A1
(43) Pub. Date: Dec. 11, 2014

(54) SURGICAL PORT LOCALIZATION

(71) Applicant: KONINKLIJKE PHILIPS N.V., EINDHOVEN (NL)

(72) Inventors: Aleksandra Popovic, New York, NY (US); Haytham Elhawary, New York, NY (US); Raymond Chan, San Diego, CA (US); Robert Manzke, Bonebuttel (DE)

(21) Appl. No.: 14/362,113

(22) PCT Filed: Nov. 12, 2012
(86) PCT No.: PCT/IB2012/056350

§ 371 Date: May 30, 2014

Related U.S. Application Data
(60) Provisional application No. 61/566,614, filed on Dec. 3, 2011.

(51) Int. Cl. A61B 1/04; A61B 1/00

(52) U.S. Cl. CPC A61B 1/04; A61B 1/00147
USPC 348/65

(57) ABSTRACT

A method, device, and system are provided for placing a port (12, 22, 32) for a surgical tool (20, 30) relative to real-time anatomical data. The method comprises: placing an endoscope (10) in a standard port (12); determining real-time anatomical data from an image from the endoscope; using a port localization apparatus (210) to identify an optimal location for an instrument port relative to the image from the endoscope; and creating an instrument port at the identified location.

### FIELD OF THE INVENTION

The invention relates to the field of medical imaging and more particularly to an apparatus and method for locating a surgical port with respect to an endoscopic device.

### BACKGROUND

Minimally invasive surgery is performed using elongated instruments 10, 20, 30 inserted into a patient's body through small ports 12, 22, 32, as shown in FIG. 1. The number of ports may vary from one port to three or more ports. In many procedures one of the instruments inserted into a patient's body is an endoscope, providing real-time visualization of a body cavity where the procedure is performed. Placement of the ports plays an important role in the outcome of the surgery. Instruments positioned in a port at a suboptimal location port may not be able to reach all areas of interest.

In a standard clinical practice, the ports are located using guidelines published by professional associations, hospitals, or manufacturers of surgical equipment. The guidelines define entry points with respect to some well-known anatomical landmarks. For example, in cardiac surgery ports are located by defining anatomical landmarks, such as: ribs, sternum, and nipple, together with defining distances from these landmarks. This kind of guidance does not take into account variability in anatomy (both between patients and between pre-operative versus intra-operative anatomy), patient size, specificity of patient's disease or condition, and the like. A more patient-specific port placement can be beneficial, minimizing access-related tissue trauma and procedure times while improving outcomes.

After the instruments are inserted into the body cavity through the ports, they can be moved in four degrees of freedom (DOF): two angles of rotation pivoting around the port (fulcrum point), insertion, and rotation about the instruments longitudinal axis. The angles of rotation are rotation about imaginary axes that are perpendicular to each other and to the longitudinal axis of the instrument. Whereas rotation and insertion are intuitive and easy to use, mapping between the angles of rotation and an endoscope view of the body cavity is less intuitive, takes a long time to learn, and, as studies show, is the biggest hurdle to hand-eye coordination in minimally invasive surgery.

Much of the research on improving port placement is focused on developing computer algorithms to compute optimal ports for instruments based on preoperative 3D medical images. The subsequent translation of these optimized plans into an operating room is focused on using standard tool tracking technologies known from surgical navigation. However, tool tracking on preoperative 3D images does not take into account that spatial relationships between anatomical landmarks may significantly change from preoperative images to intraoperative situation. For example, in minimally invasive surgery CO2 is introduced into the natural cavity (such as chest or abdomen) to expand the patient and provide a larger space for instruments. After introduction of the CO2, the spatial arrangement between outer surface of the patient (e.g. chest) and organs (such as heart) would change. Thus, the preoperative planning of port placement would not be useful in the new arrangement.

### SUMMARY

A method, device, and system are provided for placing a port for a surgical tool relative to real-time anatomical data

According to one aspect of the present invention, a method is provided for placing a port for a surgical tool relative to real-time anatomical data. The method comprises the steps of: placing an endoscope in a standard port, determining real-time anatomical data from an image from the endoscope, using a port localization apparatus to identify an optimal location for an instrument port relative to the image from the endoscope, and creating an instrument port at the identified location.

According to one embodiment, the port localization apparatus is affixed to the endoscope at a predetermined point, and the step of using a port localization apparatus to identify an optimal location for an instrument port comprises the steps of: locating a potential port location, determining a virtual projection of an instrument through the potential port location onto the plane of the endoscope image, overlaying a virtual representation of the instrument onto the endoscope image corresponding to the potential port location, and receiving an indication of whether or not the potential port location is an optimal port location.

According to one the indication of whether or not the potential port location is an optimal port location is provided by a user of the port localization apparatus.

According to one embodiment, the indication of whether or not the potential port location is an optimal port location is determined and provided by a processing device based on the position of the overlaid virtual representation of the instrument.

According to one embodiment, the method further comprises the step of: manipulating a positioning and orientation apparatus to project the virtual projection of the instrument onto the endoscope image. The positioning and orientation apparatus captures the angles of projection and a port localization program of instruction executed by a processor determines the location of the virtual projection on the endoscope image corresponding to the captured angles.

According to one embodiment, the port localization apparatus is a shape sensing tether and the positioning and orientation apparatus is a stylus. The shape sensing tether provides the 3D form of the tether so that a port localization program of instruction can determine the location of the free end of the tether in endoscope coordinates. Then a modeling program of instruction projects a virtual tool through a potential port location at the free end of the tether onto an endoscope image. The stylus allows a surgeon orient the stylus like a tool or even to manipulate it like a tool so that the orientation and the range of motion for a tool is projected onto the endoscope image. The tool orientation and the range of motion may be used to determine whether or not a potential port location is optimal.

According to one embodiment, the port localization apparatus is at least one rigid member and the positioning and orientation apparatus is at least one joint connected to the at least one rigid member and having an encoder measuring an angle of the joint.

According to another aspect of the present invention, a device is provided for locating a port for a surgical tool relative to real-time anatomical data from an endoscope. The device comprises: a port localization apparatus affixed to the endoscope at a predetermined point and locating a port at a known location relative to the endoscope.

According to one embodiment, the device further comprises a processor, the processor: determines the port location in an image space of the endoscope, determines a projection of an instrument through the port location onto an image from the endoscope, and overlaying a virtual representation of the instrument onto the endoscope image corresponding to the potential port location.

According to one embodiment the device further comprises a positioning and orientation apparatus operably connected to the port localization apparatus. The positioning and orientation apparatus is adapted to be manipulated to providing angles of projection at the port location for a projection of an instrument onto the endoscope image. The positioning and orientation apparatus captures the angles of projection and determining the location of the projection on the endoscope image.

According to one embodiment, the port localization apparatus is a shape sensing tether and the positioning and orientation apparatus is a stylus.

According to one embodiment, the port localization apparatus is at least one rigid member and the positioning and orientation apparatus is at least one joint connected to the at least one rigid member and having an encoder measuring an angle of the joint.

According to another aspect of the present invention, a system is provided for locating a port for a surgical tool relative to an endoscope. The system comprises: a processor, a memory operably connected with the processor, an endoscope providing an endoscope image, a port localization apparatus affixed to the endoscope at a predetermined point and locating a port at a known location relative to the endoscope, and a program of instruction encoded on the memory and executed by the processor to determine the location of the port.

According to one embodiment, the program of instruction executed by the processor: determines the port location in an image space of the endoscope, determines a projection of an instrument through the port location onto an image from the endoscope, and overlays a representation of the instrument onto the endoscope image corresponding to the potential port location.

According to one embodiment, the system further comprises a positioning and orientation apparatus operably connected to the port localization apparatus. The positioning and orientation apparatus is adapted to be manipulated to providing angles of projection at the port location for a projection of an instrument onto the endoscope image. The positioning and orientation apparatus captures the angles of projection and the port localization program of instruction determines the location of the projection on the endoscope image.

According to one embodiment, the port localization apparatus is a shape sensing tether and the positioning and orientation apparatus is a stylus.

According to one embodiment, the port localization apparatus is at least one rigid member and the positioning and orientation apparatus is at least one joint connected to the at least one rigid member and having an encoder measuring an angle of the joint.

### BRIEF DESCRIPTION OF THE DRAWINGS

The features and advantages of the invention will be more clearly understood from the following detailed description of the preferred embodiments when read in connection with the accompanying drawing. Included in the drawing are the following figures:

FIG. 1 is an isometric view of a minimally invasive procedure according to the prior art;

FIG. 2 is a sectional view of a minimally invasive procedure using a device for locating a port for a surgical tool relative to real-time anatomical data from an endoscope according to an embodiment of the present invention;

FIG. 3 is an endoscope image from the procedure of FIG. 2;

FIG. 4 is a block diagram of a system for locating a port for a surgical tool relative to real-time anatomical data from an endoscope according to an embodiment of the present invention;

FIG. 5 is a flow diagram of a method for locating a port for a surgical tool relative to real-time anatomical data from an endoscope according to an embodiment of the present invention;

FIG. 6 is a flow diagram of a method for using a port localization apparatus to identify an optimal location for a port for a surgical instrument relative to real-time anatomical data according to an embodiment of the present invention;

FIG. 7 is a representation of a device for locating a port for a surgical tool relative to real-time anatomical data from an endoscope according to an embodiment of the present invention;

FIG. 8 is side view of a device for locating a port for a surgical tool relative to real-time anatomical data from an endoscope according to another embodiment of the present invention;

FIG. 9 is a front view of the device of FIG. 8; and

FIG. 10 is a representation of the device of FIGS. 8 and 9 showing an alternative attachment to the endoscope according to an embodiment of the present invention.

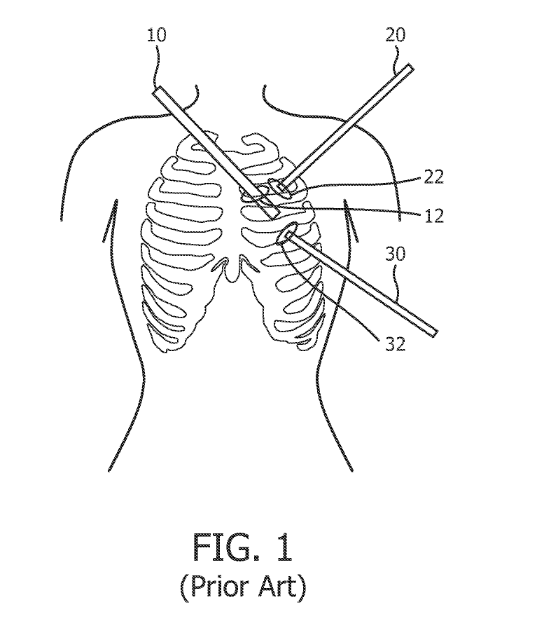

### DETAILED DESCRIPTION

The present invention provides a method, device, and system for locating a port for a surgical tool relative to real-time anatomical data from an endoscope.

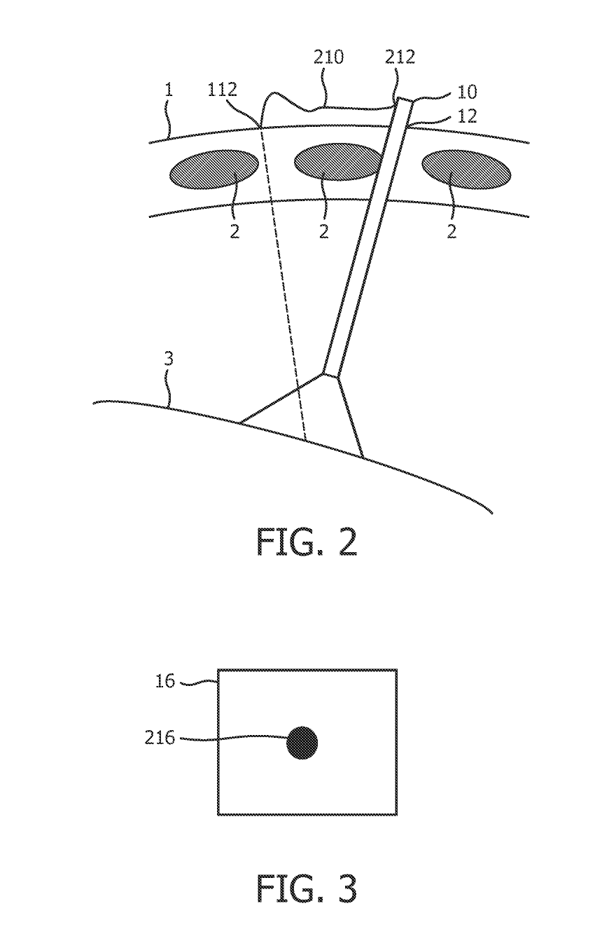

FIG. 2 shows a device for locating a port for a surgical tool relative to real-time anatomical data from an endoscope. According to one embodiment, an endoscope 10 is inserted through a standard port 12. The standard port is located using existing techniques. The endoscope provides an image 216 of anatomical features. In the illustrated embodiment, the endoscope 10 is inserted through the port 212 in the skin 1, between the ribs 2, and into the thoracic cavity to provide an image 16 of the heart 3, as shown in FIG. 3.

Returning to FIG. 2, an optical shape sensing fiber 210 (OSS) is connected to the endoscope 10 at a known anchor point 212. In shape sensing, properties of a fiber optic cable are used to determine the 3-dimensional shape of the fiber optic cable. Shape sensing based on fiber optics is known in the art. One approach is based on fiber optic Bragg grating sensors. A fiber optic Bragg grating (FBG) is a short segment of optical fiber that reflects particular wavelengths of light and transmits other wavelengths of light. This is achieved by adding a periodic variation of the refractive index in the fiber core, which generates a wavelength-specific dielectric mirror. An FBG can therefore be used as an inline optical filter to block transmission of certain wavelengths or as a selective specific wavelength reflector—the specific wavelength being the Bragg wavelength.

The Bragg wavelength is sensitive to strain as well as temperature. This means that Bragg gratings can be used as sensing elements in fiber optic sensors. In an FBG sensor, the measurand (e.g., strain) causes a shift in the Bragg wavelength.

One of the main advantages of FBG's is that various sensor elements can be distributed over the length of a fiber. Incorporating 3 or more cores with various sensors (gauges) along the length of a fiber that is embedded in a structure allows for the 3-dimensional form of the structure to be precisely determined. A plurality of FBG sensors are positioned along the length of the fiber (e.g., in 3 or more sensing cores). From the Bragg wavelength shift, the strain can be measured at each FBG. From the strain measurements, at various positions, the curvature at these positions can be inferred. From the plurality of measured positions, the total 3-dimensional form is determined.

As an alternative to FBG, the inherent backscatter in conventional optical fiber can be exploited for shape sensing. One such approach is to use Raleigh scatter in standard, single-mode communications fiber. Raleigh scatter occurs as a result of random fluctuations of the index of refraction in the fiber core. These random fluctuations can be modeled as a Bragg grating with random variation of amplitude and phase along the grating length. By using this effect in 3 or more cores running within a single length of multicore fiber, the 3D shape and dynamics of the surface of interest is trackable.

With the optical fiber connected to the endoscope at a known anchor point, the spatial relationship to the endoscope image 16 is established using two mechanical properties of the mounting: (1) the distance from the center axis of the endoscope to the anchor point (d); and (2) the distance from the anchor point to the endoscope image plane (l) (i.e., the focal length of the endoscope+the distance from the lens of the endoscope to the anchor point along the axis of the endoscope).

These two values: (d) and (l) are used to translate the coordinate system of the fiber to the coordinate system of the endoscopic image so that the measured shape of the optic fiber beyond the anchor point 212 can be calculated in the endoscope coordinate system. Thus, a potential port location 112 can be contacted with the free end of the optic fiber 210 to locate it relative to the endoscopic image 16. Moreover, the shape of the optic fiber can be extended using a 3D modeling application to project a virtual surgical tool onto the plane of the endoscopic image 16. A representation of the surgical tool 216, such as a virtual dot, is then overlaid onto the endoscopic image 16, and used to determine whether or not the proposed port location 112 is optimal.

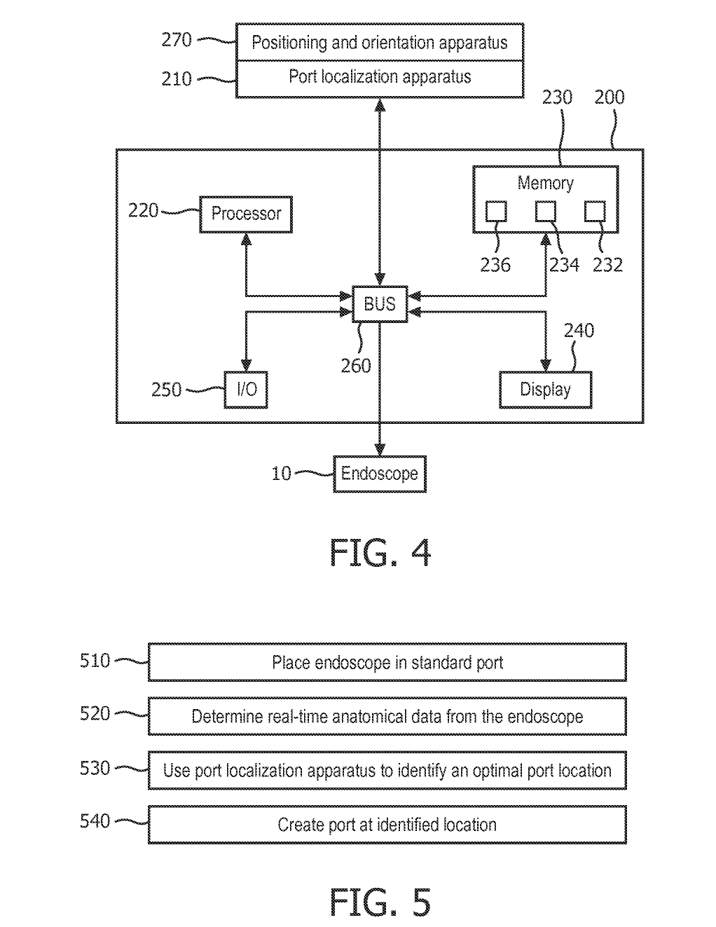

FIG. 4 is a block diagram of a system for placing a port for a surgical tool relative to real-time anatomical data according to an embodiment of the present invention. The system comprises: a port localization apparatus 210, a processing system 200, and an endoscope 10. The processing system 200 comprises one or more processors 220 operably connected with one or more memories 230. They may be connected, for example, through a system bus 260. It should be understood that other suitable architectures are also possible within the scope of the present invention. The processor 220 may be any suitable processor, such as one or more microprocessors. The memory 230 may be any suitable memory, including but not limited to: RAM, ROM, an internal hard drive, a disk drive, a USB flash drive, or any other memory device suitable for storing program code.

The memory 230 has encoded on it a localization program of instruction 232 executed by the processor 220 to locate a port for a surgical tool relative to real-time anatomical data from the endoscope 10. The system also comprises one or more displays 240 for presenting a user interface with endoscope data and one or more input and/or output devices 250 for entering user inputs, for example.

The memory 230 may also have endoscope program instructions 234 for presenting an endoscope image 16 on the display 240, and modeling program instructions 236 for modeling and projecting a virtual representation of a surgical tool through a potential port location onto the endoscope image 16.

FIG. 5 is a flow diagram of a method for locating a port for a surgical tool relative to real-time anatomical data from an endoscope according to an embodiment of the present invention. A surgeon places an endoscope 10 in a standard port 12 (Step 510). That is, the surgeon locates the port using a standard port locating procedure.

The processor 220 executes the endoscope program instructions 232 to determine real-time anatomical data from the endoscope 10 (Step 520). The real-time anatomical data may be the digitized endoscopic image 16, such as the coordinates and intensity values for each pixel of the image.

The port localization program instructions 232 executed by the processor 220 uses the port localization apparatus 210 to identify an optimal port location (Step 530). Then, the surgeon creates a port at the determined location (Step 540).

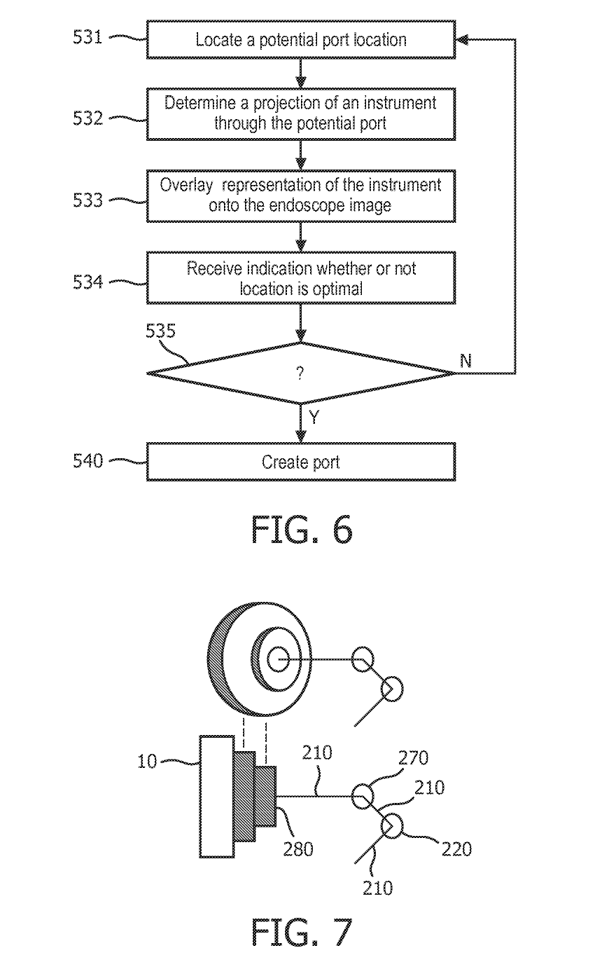

According to one embodiment, the port localization apparatus 210 comprises a tether with an optical shape-sensing fiber which is fixed to the endoscope at a known anchor point 212. As shown in FIG. 6, the free end of the optical shape-sensing fiber is positioned at a potential port location (Step 531). Then, the port localization program instructions 232 executed by the processor 220 projects a virtual tool through the potential port location onto the endoscopic image 16 (Step 532). The virtual tool is projected by translating the location of the potential port to endoscopic image coordinates, then extending a line from the potential port location onto the endoscopic image. The line is extended at predetermined tool angles pivoted about the potential port location, such as parallel to the endoscope, perpendicular to the surface of the patient's skin, or at any other predetermined angle. The projection is used to determine whether or not the potential port location is optimal. This determination may be made by the surgeon or by the port localization program instructions 232 executed by the processor 220.

If the potential port location is optimal, then the surgeon creates a port at the determined location (Step 540).

According to one embodiment, a representation of the projection of the surgical tool is overlaid on the endoscopic image 16 (Step 533). Based on the position of the representation, a determination is made of whether or not the potential port location is optimal. This determination can be made by the surgeon after looking at the position of the representation of the tool, or by the port localization program instructions 232 executed by the processor 220 using a modeling application. The port localization program instructions 232 executed by the processor 220 then receives the indication of whether or not the location is optimal (Step 534). The port localization program instructions 232 executed by the processor 220 may iteratively test potential port locations until an optimal location is located (Step 535).

According to another embodiment, a virtual line is determined directly from the free end of the optic fiber 210 to the center of the endoscope image 16 and the resulting tool angles at the potential port location are determined using a modeling application. Then a determination of whether or not the potential port location is optimal is made based on the determined tool angles.

According to one embodiment, a positioning and orientation apparatus 270 is connected to the localization apparatus 210 to project the projection of the instrument onto the endoscope image 16. The positioning and orientation apparatus 270 captures the angles of projection and determines the location of the projection on the endoscope image corresponding to the captured angles. The poisoning and orientation apparatus 270 may be, for example, a stylus simulating the handle of a surgical tool. The optic fiber 210 may be fixed in the stylus, such as through a longitudinal opening, such that the end of the optic fiber (captured in the stylus) is held at the tool angles relative to the potential port location. In this embodiment, the angles are captured by shape sensing of the optic fiber.

The surgeon holds the positioning and orientation apparatus 270 at a potential port location and manipulates it to simulate the intended procedure. As described in the previous embodiment, the port locating program of instruction 232 translates the potential port location to endoscope coordinates using the known anchor point and the determined shape of the optic fiber. The modeling program instructions 236 extend a virtual tool from the potential port location along the angles defined by the end of the optic fiber held in the stylus. The projection of the tool is then overlaid onto the endoscope image 16 so that the surgeon can see the range of motion corresponding to the potential port location and determine whether or not the potential port location is optimal.

FIG. 7 shows a representation of a device for locating a port for a surgical tool relative to real-time anatomical data from an endoscope according to another embodiment of the present invention. In this embodiment, the port localization apparatus 210 is at least one rigid member such as an arm, a beam, or the like. The positioning and orientation apparatus 270 is at least one hinged joint with an encoder that provides the angle of the joint. The angle can be provided as an electronic signal or it can be read off of the encoder and entered manually to the port location program instructions 232. As with the optic fiber, the rigid port localization apparatus 210 has a known anchor point on the endoscope. Also, the length of the port localization apparatus 210 is known. The modeling program instructions can determine the location of the end of the port localization apparatus, and the potential port location, from the known anchor point, the arm length, and the joint angles.

According to one embodiment, the joints 270 are motorized to manipulate a port localization apparatus, or even a surgical tool, during a procedure by driving the joints to angles corresponding to a desired location for automatic positioning. In this embodiment, the surgeon may input a location on the endoscope image to automatically position a tool.

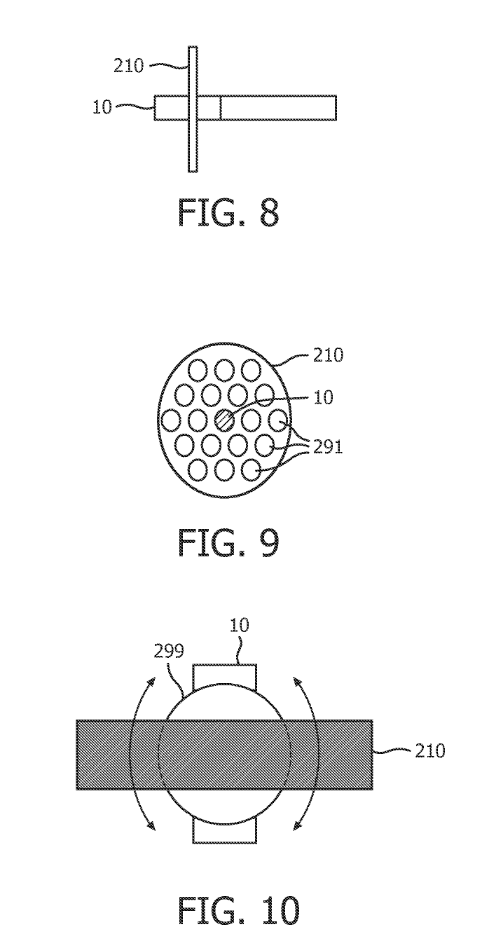

FIGS. 8 and 9 show a port localization apparatus 210 according to another embodiment of the present invention. In this embodiment, the port localization apparatus 210 is a rigid disk mounted on the endoscope 10 at a center hole. The disk is provided with a plurality of holes 291 which are numbered. Each hole locates a potential port location. Because the disk is rigid, and the holes are a known distance from the center of the endoscope at a known orientation, the holes 291 can be translated into endoscope coordinates, and virtual tools can be projected from the corresponding potential port locations onto the endoscopic image 16. Representations of these virtual tools, such as numbers corresponding to the numbered holes are then overlaid onto the endoscope image 16. An optimal port location may then be selected based on the location of the corresponding virtual tool projection.

As shown in FIG. 10, the disk port localization apparatus 210 may be attached to the endoscope with a spherical joint 299 to allow the disk to rotate relative to the endoscope 10 so that the virtual tools may be projected at an angle to the potential port location. The spherical joint may be equipped with an encoder to provide the angle of rotation of the disk.

The invention can take the form of an entirely hardware embodiment or an embodiment containing both hardware and software elements. In an exemplary embodiment, the invention is implemented in software, which includes but is not limited to firmware, resident software, microcode, etc.

Furthermore, the invention may take the form of a computer program product accessible from a computer-usable or computer-readable medium providing program code for use by or in connection with a computer or any instruction execution system or device. For the purposes of this description, a computer-usable or computer readable medium may be any apparatus that can contain or store the program for use by or in connection with the instruction execution system, apparatus, or device.

The foregoing method may be realized by a program product comprising a machine-readable medium having a machine-executable program of instructions, which when executed by a machine, such as a computer, performs the steps of the method. This program product may be stored on any of a variety of known machine-readable medium, including but not limited to compact discs, floppy discs, USB memory devices, and the like.

The medium can be an electronic, magnetic, optical, electromagnetic, infrared, or semiconductor system (or apparatus or device). Examples of a computer-readable medium include a semiconductor or solid state memory, magnetic tape, a removable computer diskette, a random access memory (RAM), a read-only memory (ROM), a rigid magnetic disk an optical disk. Current examples of optical disks include compact disk-read only memory (CD-ROM), compact disk-read/write (CD-R/W) and DVD.

The preceding description and accompanying drawing are intended to be illustrative and not limiting of the invention. The scope of the invention is intended to encompass equivalent variations and configurations to the full extent of the following claims.

1. A method for placing a port for a surgical tool relative to real-time anatomical data, comprising the steps of:
placing an endoscope in a standard port;
determining real-time anatomical data from an image from the endoscope;
using a port localization apparatus to identify an optimal location for an instrument port relative to the image from the endoscope; and
creating an instrument port at the identified location.

2. The method of claim 1, wherein the port localization apparatus is affixed to the endoscope at a predetermined anchor point, and wherein the step of using a port localization apparatus to identify an optimal location for an instrument port comprises the steps of:
locate a potential port location;
determining a projection of an instrument through the potential port location onto the plane of the endoscope image;
overlaying a representation of the instrument onto the endoscope image corresponding to the potential port location; and
receiving an indication of whether or not the potential port location is an optimal port location.

3. (canceled)

4. (canceled)

5. The method of claim 2, further comprising the step of:
manipulating a positioning and orientation apparatus to project the projection of the instrument onto the endoscope image, the positioning and orientation apparatus capturing the angles of projection and determining the location of the projection on the endoscope image corresponding to the captured angles.

6. (canceled)

7. (canceled)

8. (canceled)

9. A device for locating a port for a surgical tool relative to real-time anatomical data from an endoscope, comprising:
an endoscope; and
a port localization apparatus affixed to the endoscope at a predetermined anchor point
characterized in the port localization apparatus defines a location for a port at a known spatial relationship to the endoscope.

10. The device of claim 9, further comprising a processor, the processor:
determining the port location in an image space of the endoscope;
determining a projection of an instrument through the port location onto an image from the endoscope; and
overlaying a representation of the instrument onto the endoscope image corresponding to the potential port location.

11. The device of claim 10, further comprising a positioning and orientation apparatus operably connected to the port localization apparatus, the positioning and orientation apparatus being adapted to be manipulated to providing angles of projection at the port location for a projection of an instrument onto the endoscope image, the positioning and orientation apparatus capturing the angles of projection and determining the location of the projection on the endoscope image.

12. The device of claim 11, wherein the port localization apparatus is a shape sensing tether and the positioning and orientation apparatus is a stylus.

13. The device of claim 10, wherein the port localization apparatus is at least one rigid member and the positioning and orientation apparatus is at least one joint connected to the at least one rigid member and having an encoder measuring an angle of the joint.

14. A system for locating a port for a surgical tool relative to an endoscope, comprising:
a processor;
a memory operably connected with the processor;
an endoscope providing an endoscope image;
a port localization apparatus affixed to the endoscope at a predetermined anchor point; and locating a port at a known location relative to the endoscope; and
a program of instruction encoded on the memory and executed by the processor;
characterized in the port localization apparatus defines a port at a known spatial relationship relative to the endoscope; and
the program of instruction encoded on the memory and executed by the processor determines the location of the port.

15. The system of claim 14, wherein the program of instruction executed by the processor:
determines the port location in an image space of the endoscope;
determines a projection of an instrument through the port location onto an image from the endoscope; and
overlays a representation of the instrument onto the endoscope image corresponding to the potential port location.

16. The system of claim 15, further comprising a positioning and orientation apparatus operably connected to the port localization apparatus, the positioning and orientation apparatus being adapted to be manipulated to providing angles of projection at the port location for a projection of an instrument onto the endoscope image, the positioning and orientation apparatus capturing the angles of projection and determining the location of the projection on the endoscope image.

17. The system of claim 14, wherein the port localization apparatus is a shape sensing tether and the positioning and orientation apparatus is a stylus.

18. The system of claim 14, wherein the port localization apparatus is at least one rigid member and the positioning and orientation apparatus is at least one joint connected to the at least one rigid member and having an encoder measuring an angle of the joint.

19. A computer program product comprising a computer-readable storage device having encoded thereon a program of instruction executable by a computer processor to place a port for a surgical tool relative to real-time anatomical data, the program of instruction comprising:
program instructions for determining real-time anatomical data from an image from an endoscope;
characterized in the program of instructions further comprising program instructions for using a port localization apparatus to identify an optimal location for an instrument port relative to the image from the endoscope.

20. The computer program product of claim 19, further comprising:
program instructions for locate a potential port location;
program instructions for determining a projection of an instrument through the potential port location onto the plane of the endoscope image;
program instructions for overlaying a representation of the instrument onto the endoscope image corresponding to the potential port location; and
program instructions for receiving an indication of whether or not the potential port location is an optimal port location;
wherein the program instructions are encoded on the computer-readable storage device.
